(19) United States
(12) Patent Application Publication
(10) Pub. No.: US 2005/0079236 A1
(43) Pub. Date: Apr. 14, 2005

(54) Citrus-derived cosmetic and medicinal composition, and nutritional food ingredient and associated methods of application

(76) Inventor: Jason Robert Ahrens, Lake Alfred, FL (US)

Correspondence Address: BEUSSE BROWNLEE WOLTER MORA & MAIRE, P. A., 390 NORTH ORANGE AVENUE, SUITE 2500, ORLANDO, FL, 32801, US

(21) Appl. No.: 10/928,918

(22) Filed: Aug. 27, 2004

Related U.S. Application Data
(60) Provisional application No. 60/498,219, filed on Aug. 27, 2003.

(51) Int. Cl. A61K 35/78

(52) U.S. Cl. 424/736

(57) ABSTRACT

A method to encompass a citrus-based cream or gel composition that is highly stable and all-natural from components in situ by removing undesired components from citrus, producing such cream or gel with desired components and texture according to intended use, either as a cosmetic or medicinal lotion, a cream base, or as a food ingredient with nutraceutical and/or binding properties for use in as example analogue meats. The method comprises the steps of grinding whole citrus fruit or peel adjusted via rate of heating and pH to optimize gel consistency and desired residual bioflavonoids rather than maximizing pectin production. Citrus-derived components such as, but not limited to, d-limonene, naringin, and psoralen are added to and blended with the gel for applications which require concentrations higher than found in vivo.

### BACKGROUND OF THE INVENTION

1. Field of the invention

The present invention relates to compositions with inherent specific textures for cosmetic and medicinal uses and for use as a nutritional food ingredient, more particularly, to such compositions that are made from all-natural ingredients primarily in situ, and most particularly, citrus.

2. Description of Related Art

Compounds found primarily in the peel portion of citrus, but nonetheless throughout the fruit, are known to have anti-histamine and anti-inflammatory (Ko H H et al. Anti-inflammatory flavonoids and pterocarpanoid from Crotalaria pallida and C. assamica. Bioorg Med Chem Lett. Feb. 23, 2004; 14(4):1011-4. Souza M C et al. Evaluation of anti-inflammatory activity of essential oils from two Asteraceae species. Pharmazie. August 2003; 58(8):582-6. Rotelli A E et al. Comparative study of flavonoids in experimental models of inflammation. Pharmaco Res. December 2003; 48(6):601-6. Theoharides T C et al. Anti-inflammatory actions of flavonoids and structural requirements for new design. Int J Immunopathol Pharmacol. September-December 2001; 14(3):119-127. Wallace J M. Nutritional and botanical modulation of the inflammatory cascade—eicosanoids, cyclooxygenases, and lipoxygenases—as an adjunct in cancer therapy. Integr Cancer Ther. March 2002; 1(1):7-37.), anti-microbial (Moura I C et al. Limoriene arrests parasite development and inhibits isoprenylation of proteins in Plasmodium falciparum. Antimicrob Agents Chemother. September 2001; 45(9):2553-8. Rauha J P et al. Antimicrobial effects of Finnish plant extracts containing flavonoids and other phenolic compounds. Int J Food Microbiol. May 25, 2000; 56(1):3-12.), anti-oxidant (Kim H J et al. Naringin alters the cholesterol biosynthesis and antioxidant enzyme activities in LDL receptor-knockout mice under cholesterol fed condition. Life Sci. Feb. 13, 2004; 74(13):1621-34. Jung U J et al. Naringin supplementation lowers plasma lipids and enhances erythrocyte antioxidant enzyme activities in hypercholesterolemic subjects. Clin Nutr. December 2003; 22(6):561-8. Russo A et al. Bioflavonoids as antiradicals, antioxidants and DNA cleavage protectors. Cell Biol Toxicol. 2000; 16(2):91-8. Lee K G et al. Inhibitory effects of plant-derived flavonoids and phenolic acids on malonaldehyde formation from ethyl arachidonate. J Agric Food Chem. Nov. 19, 2003:51 (24):7203-7), and anti-toxic shock properties (Kawaguchi K et al. Suppression of infection-induced endotoxin shock in mice by a citrus flavanone naringen. Planta Med. January 2004:70(1):17-22). Grapefruit (Citrus paradise'), in particular, has been reported to contain high amounts of these compounds, among which primarily are the bioflavonoids such as apigenin, cryptoxanthin, naringin, rutin, hesperitin, quercetin, psoralen, (and their derivatives): pectin; and d-limonene. Although these compounds are found throughout nature, they are particularly concentrated in citrus, especially grapefruit. In addition, certain have been shown to promote healthy skin (Boelsma E et al. Human skin condition and its associations with nutrient concentrations in serum and diet. Am J Clin Nutr. February 2003; 77(2):348-55.), protect against UV radiation damage (Proteggente A R et al. Hesperetin glucuronide, a photoprotective agent arising from flavonoid metabolism in human skin fibroblasts. Photochem Photobiol. September 2003; 78(3):256-61.), stabilize DNA (Russo A et al. Bioflavonoids as antiradicals, antioxidants and DNA cleavage protectors. Cell Biol Toxicol. 2000;16(2):91-8.), and promote wound healing (Phan T T et al. Dietary compounds inhibit proliferation and contraction of keloid and hypertrophic scar-derived fibroblasts in vitro: therapeutic implication for excessive scarring. J Trauma. June 2003; 54(6):1212-24. Phan T T et al. Suppression of insulin-like growth factor signaling pathway and collagen expression in keloid-derived fibroblasts by quercetin: its therapeutic potential use in the treatment and/or prevention of keloids. Br J Dermatol. March 2003; 148(3):544-52.). Pectin hydrogels, with and without anti-microbial agents, are used as a wound dressing (Shalimov S A et al. The use of pectin hydrogel in the therapy of acute postradiation epitheliitis and trophic disorders. Kiln Khir. 1995; (3):26-7.). Psoriasis affects 3% of all Americans. Research by the National Psoriasis Foundation has indicated it may be treated with a combination of psoralen and UVA radiation.

Cream bases are used in a wide variety of cosmetic and medicinal compositions. Such cream bases have mainly included aloe, lanolin, or synthetic carriers such as petroleum. Lanolin has been found to be an allergen in a segment of the population, and aloe alone has not been successfully used to make cream bases. Petroleum carriers are often avoided, since current market demand includes a desire for more all-natural products, as well they are not water soluble. Pectin is the primary base for commercial gels used in jellies and as body agents in beverages. When properly prepared, it produces a natural gel ideal for use as a cream base.

In addition to topical treatment, bioflavonoids and pectin have been shown to reduce cholesterol (Kim H J et al. Naringin alters the cholesterol biosynthesis and antioxidant enzyme activities in LDL receptor-knockout mice under cholesterol fed condition. Life Sci. Feb. 13, 2004; 74(13):1621-34. Kim H K et al. Lipid-lowering efficacy of hesperetin metabolites in high-cholesterol fed rats. Clin Chim Acta. January 2003; 327(1-2):129-37.) and lower blood lipid levels and uptake (Jung U J et al. Naringin supplementation lowers plasma lipids and enhances erythrocyte antioxidant enzyme activities in hypercholesterolemic subjects. Clin Nutr. December 2003; 22(6):561-8. Tsujita T et al. Inhibition of lipase activities by citrus pectin. J Nutr Sci Vitaminol (Tokyo). October 2003; 49(5):340-5. Han L K et al. Anti-obesity action of Salix matsudana leaves (Part 2). Isolation of anti-obesity effectors from polyphenol fractions of Salix matsudana. Phytother Res. December 2003; 17(10):1195-8.).

### SUMMARY OF THE INVENTION

The present invention addresses the need for an all-natural cream base, specifically a gel, produced from citrus components in situ that has medicinal properties in and of itself and also can serve as a base for cosmetics such as moisturizers; and a further need for a food ingredient which has nutraceutical properties in and of itself and also can serve as a texturizer or binder in food products such as sausages and analogue meats.

The method of the present invention is intended to encompass the citrus-based cream or gel composition that is highly stable and all-natural from components in situ by removing undesired components from citrus, producing such cream or gel with desired components and texture according to intended use, either as a cosmetic or medicinal lotion, a cream base, or as a food ingredient with nutraceutical and/or binding properties for use in as example analogue meats. The method comprises the steps of grinding whole citrus fruit or peel (hereinafter referred to as peel, but not excluding whole fruit) into a puree consistency and using a method or combination of methods known to one skilled in the art such as detailed by Jameson, Taylor and Wilson (1924. Pectin Product and Process of Producing Same. U.S. Pat. No. 1,497,884), Kirk and Othmer (1957. Pectic Substances./Pectin Manufacture. pp 908-916. In: Encyclopedia of Chemical Technology. Kirk, R and Othmer, D. (eds.) The Interscience Encyclopedia, Inc. NY) or Graham (1977. Commercially Important Pectic Substances/Manufacture. pp 425-430. In: Food Colloids. AVI Publishing Co Westport, Conn.) adjusted via rate of heating and pH to optimize gel consistency and desired residual bioflavonoids rather than maximizing pectin production. Citrus-derived components such as, but not limited to, d-limonene, naringin, and psoralen are added to and blended with the gel for applications which require concentrations higher than found in vivo.

The invention is also intended to encompass the application of such citrus-based cream or gel for cosmetic or medicinal use, as a base, or with cosmetic and medicinal properties in and of itself. Such citrus-based cream or gel is useful for the treatment of psoriasis and age-spots, dandruff, rosacea, inflammation, wound healing, sunburn and other UV damage, melanoma, fungal and yeast infections including but not limited to tinea infections and chlamydia, wrinkles, as a moisturizer, skin sensitizer, skin lubricant, and skin sanitizer.

The invention is also intended to encompass the application of such citrus-based cream or gel as a food ingredient with nutraceutical and/or binding properties. Such citrus-based cream or gel is useful to serve as a binder for meat products and analogue meat products such as sausages, patties, and loaves, for example to replace or enhance food grade gums. As a food ingredient, it is useful to serve as an anti-oxidant, a source of dietary fiber, and a cholesterol and lipid-lowering nutraceutical, enhancing a food's functionality.

Grapefruit is preferred for the production of this gel because of its high concentration of active compounds. It should be noted however, that this treatment may be utilized with all species of citrus and is not intended to be limited to grapefruit. Different species of citrus can be used separately or in a mixture to achieve an inventive result. The active components of citrus for this treatment are numerous and are believed to work in concert to achieve the desired result. The compounds are found in high concentrations in the peel of the fruit but may be found in the juice, seed, and rag (membranes) portions as well.

The features that characterize the invention, both as to organization and method of operation, together with further objects and advantages thereof, will be better understood from the following description.

### DETAILED DESCRIPTION OF THE PREFERRED EMBODIMENTS

A description of the preferred embodiments of the present invention will now be presented.

No claim is made as to the manufacturing technique or method of extraction to produce pectin which is incorporated into the gel. Any one of several pectin or gel production techniques known to one skilled in the art is appropriate. The exception is that acid used to adjust pH must be on the US Food and Drug Generally Recognized As Safe (GRAS) list and acceptable for use in food products such as acetic acid. The method of the present invention is intended to encompass the citrus-based cream or gel composition that is highly stable and all-natural. The novelty is that the resulting gel has a composition derived from components naturally in situ in citrus fruit by removing undesired components from citrus, producing such cream or gel with desired components and texture according to intended use, either as a cosmetic or medicinal lotion, a cream base, or as a food ingredient with nutraceutical and/or binding properties for use in as example analogue meats.

With appropriate adjustments to time and temperature during the gel manufacture process, a gel can be produced which has most, or none, of the citrus bioflavonoids, citrus oils, and psoralens naturally present in untreated citrus peel. An exemplary method follows.

The method comprises the steps of squeezing the fruit via cold pressing and collecting the peel and natural oils, which are commonly separated during juicing whole fruit. The peel is ground into a puree or paste, for example using a standard size-reduction unit commonplace in the food processing industry. Particle size reduction is further achieved by adding equal parts of water and re-circulating through a food processor until a cream consistency is achieved. An exemplary embodiment of this step comprises grinding the cold-pressed peel one pass through a screw-drive Hobart commercial meat grinder using a ¼ inch output screen followed by one pass through a Comitrol food processor. The ground peel is then mixed with equal parts water and further reduced using a high-speed food processor with a micro-cut head until a cream consistency is achieved. The cream is gradually heated to near boiling to inactivate pectin methyl esterase. Three parts water is added to provide a workable solution. The pH is adjusted to between 1.2 and 3.0 with glacial acetic acid and held at 65 C. to 95 C. for 0.5 to 2.0 hours, depending on the amount of bioflavonoids desired to remain in the final gel and to remove unwanted compounds such as sugars and water soluble pectin. The combination of low pH and high temperature also causes a thin gel to form. The solution is filtered using a sock filter with a mesh size equivalent to Miracloth to remove excess water which concentrates the pectin solution, forming a gel of desired consistency. These steps are a commercial adaptation of the method outlined by Kirk and Othmer to produce liquid pectin (1957. Pectic Substances./Pectin Manufacture. pp 908-916. In: Encyclopedia of Chemical Technology. Kirk, R and Othmer, D. (eds.) The Interscience Enclyclopedia, Inc. NY). The gel can then be blended with citrus oil recovered during cold pressing in a wide range of ratios (0 to 10%) depending on the desired application. The oil also serves as a natural preservative.

It may be appreciated by one skilled in the art that additional embodiments may be contemplated, including similar manufacturing procedures for peel size reduction and gel production techniques such as alternative amounts of water, pH, temperatures, and treatment times to achieve substantially the same result: a homogenous gel derived from citrus comprised of pectin, cellulose, citrus bioflavonoids, citrus oils, and certain sugars obtained by extracting those components not desired in the final product.

Pectin, present in the gel, has been shown to aid in wound healing by serving as a scaffold on which skin cells can proliferate, as well, pectin gel keeps wounds moist. D-limonene is an effective anti-microbial agent, along with naringin and quercetin. Other bioflavonoids, such as apigenin and naringin are anti-inflammatants. In addition, bioflavonoids have been shown to protect against UV damage to skin, promote new cell growth and reduce scarring. Therefore, a citrus-based cream or gel with such composition is an all natural skin cleanser, a moisturizer, and healer for example an after shave lotion, body lotion, facial lotion, hand lotion, sunburn lotion, burn dressing, and wound dressing.

D-limonene has been shown to have strong anti-microbial activity. It is also an effective detergent. D-limonene is a T-4 lymphocyte inhibitor. Therefore, a citrus-based cream or gel with such composition is an all natural acne treatment by cleansing and sanitizing facial skin and pores, and by preventing epidermal white blood cell proliferation associated with acne.

Pectin gel is a moisturizer because of its high water content. D-limonene is a strong anti-microbial agent. Therefore, a citrus-based cream or gel with such composition is an all natural fungicide for use in the treatment of tinea infections such as athlete's foot and ringworm, as well as Chlamydia infections.

Grapefruit oil is comprised of approximately 80% d-limonene. D-limonene has been shown to reduce the incidence of melanoma. In addition bioflavonoids have been shown to be effective against a broad array of carcinomas. Naringin has been shown to stabilize DNA. Therefore, a citrus-based cream or gel with such composition is an all natural agent against skin cancers.

Elastase, an enzyme in the connective tissue under the skin, destroys elastin which is implicated as a leading cause of wrinkles. D-limonene has been shown to penetrate the skin and prevent the activity of elastase. Therefore, a citrus-based cream or gel with such composition is an all natural anti-wrinkle cream.

Psoriasis is a disease affecting 3% of the population. It is associated with inflammation, histamine, and over production of dermai cells. It can exist as mild red spots on the skin, to excessively thick bleeding patches of skin. There are no known cures for psoriasis. However, in many cases an effective treatment is administration of psoralen combined with UV A radiation. Some forms of dandruff are related to psoriasis. Grapefruit oil contains psoralen. Bioflavonoids have anti-histamine and anti-inflammatory properties. Therefore, a citrus-based cream or gel with such composition is an all natural treatment for psoriasis and dandruff.

Pectin forms a gel with specific water content, according to the temperature, time, and pH of manufacture. Because of its unique hydrostatic and hydrogen bonding capacity, it can be used as a binding agent with most blended and mixed food, but specifically, for meat and meat analogue sausages, patties, and loaves, It can replace and or enhance gums in such foods. Its advantage is that the water content and thus the binding ability can be varied according to method of manufacture. Therefore, a citrus-based cream or gel with such composition is an effective all natural binder in foods such as meat and meat analogue sausages, patties, and loaves.

Citrus pectin and citrus bioflavonoids have been shown to reduce low density lipoprotein cholesterol and to reduce blood lipid concentration and lipid uptake by fat cells. Therefore, a citrus-based cream or gel with such composition added to another food in an appropriate concentration would be an effective all natural functional food, serving to lower cholesterol and blood lipid levels.

1. A medicinal and cosmetic citrus-based cream or gel composition that is highly stable and all-natural comprised from citrus components in situ comprising pureed citrus fruit peel having been diluted with water, heated for a sufficient time and to a sufficient temperature, and pH adjusted to below 4.0 to substantially remove water-soluble pectins and undesired components and filtered to a desired gel consistency, with cosmetic or medicinal properties in and of itself, or as a cream base for cosmetic or medicinal applications.

2. A citrus-based food ingredient with binding properties for use in as example meat and meat analogue sausages, patties, and loaves, comprising pureed citrus fruit peel having been diluted with water, heated for a sufficient time and to a sufficient temperature, and pH adjusted to below 4.0 to substantially remove water-soluble pectins and undesired components and filtered to a desired gel consistency.

3. A citrus-based food ingredient with nutraceutical properties specifically to reduce low density lipoprotein cholesterol and blood lipid level and uptake by fat cells, for use in as example meat and meat analogue sausages, patties, and loaves, comprising pureed citrus fruit peel having been diluted with water, heated for a sufficient time and to a sufficient temperature, and pH adjusted to below 4.0 to substantially remove water-soluble pectins and undesired components and filtered to a desired gel consistency

4. Applications for a citrus-based cream or gel containing certain bioflavonoids, pectin, and d-limonene in certain combinations for the treatment of acne, tinea and Chlamydia infections, wrinkles, promoting wound healing, psoriasis, dandruff, minor cuts and abrasions, scarring, burns, sunburns, UV damage, melanoma, inflammations, and to serve as general skin cleansers and sanitizers, body lotions, hand lotions, facial lotions, skin and personal lubricants, after shaves, and moisturizers.
